(19) United States
(12) Patent Application Publication
(10) Pub. No.: US 2009/0304725 A1
(43) Pub. Date: Dec. 10, 2009

(54) Vaccine and Antigen Mimotopes Against Cancerous Diseases Associated with the Carcinoembryonic Antigen CEA

(75) Inventors: Erika Jensen-Jarolim, Vienna (AT); Kira Brämswig, Vienna (AT); Angelika Riemer, Vienna (AT); Christoph Zielinski, Vienna (AT); Otto Scheiner, Perchtoldsdorf (AT)

Correspondence Address: MILBANK, TWEED, HADLEY & MCCLOY LLP, INTERNATIONAL SQUARE BUILDING, 1850 K STRET, N.W., SUITE 1100, WASHINGTON, DC, 20006, US

(73) Assignee: Medizinische Universitat Wien, Vienna (AT)

(21) Appl. No.: 12/278,343

(22) PCT Filed: Feb. 5, 2007
(86) PCT No.: PCT/EP07/00967

§ 371 Date: Dec. 23, 2008

(30) Foreign Application Priority Data
Feb. 6, 2006 (EP) 06 002 369.4

(51) Int. Cl. A61K 39/00; C40B 30/04; C07K 4/12; C07K 14/47; C07K 7/06

(52) U.S. Cl. 424/185.1; 424/184.1; 506/9; 530/324; 530/325; 530/326; 530/327; 530/328; 530/329

(57) ABSTRACT

The present invention relates to a vaccine against cancerous diseases associated with the carcinoembryonic antigen CEA.

The present invention relates to a vaccine against cancerous diseases associated with the carcinoembryonic antigen CEA, the respective antigen mimotopes and the production process and use thereof.

The carcinoembryonic antigen (CEA) is a glycoprotein overexpressed by different tumours, typically by colorectal carcinoma. Additionally, elevated serum levels of CEA are found in more than 50% of all breast cancers, 70% of small cell lung carcinoma, non-small cell lung cancer, esophagus, pancreas, gastric, and thyroid carcinomas. Among all cancers, colorectal carcinoma is the second most important cause of deaths due to malignancies in the U.S.A. and other industrialized countries. This cancer occurs in male and female persons with equal incidences. Different possibilities of CEA-specific immunotherapy have been investigated so far: anti-idiotypic vaccines, CEA-pulsed dendritic cells, vaccination with recombinant CEA; DNA- and peptide vaccinations, all with varying efficacy [N. L. Berinstein, J. Clin. Oncol. 2002; 20; 2197-2207]. Carcinoembryonic antigen (CEA) represents an interesting target for anti-tumour immunotherapy as it is specifically and highly expressed by many different malignancies [Z. Qu, G. L. Griffiths, W. A. Wegener, Methods 2005; 36; 84-95]. Antibodies have so far been applied for radioimmunoscinitgraphy or radioimmunotherapy. A prominent example is the anti-CEA antibody labetuzumab, recently tested in a phase I. clinical trial [S. V. Govindan et al., J. Nucl. Med. 2005; 46; 153-159]. Direct killing effects of antibodies to tumour cells rely e.g. on ADCC (antibody dependent cellular cytotoxicity) and CDC (complement dependent cytotoxicity) reactions [R. D. Blumenthal et al., Cancer Immunol. Immunother. 2005; 54; 315-327].

Major disadvantages of passive immunotherapies are that antibodies have to be repeatedly applied intravenously and given at high doses to achieve the desired tissue distribution and clinical effects. This means that their practical application in the individual patient is limited by the costs of manufacturing.

Moreover, CEA has been found to exhibit only a low immunogenicity due to its 50% carbohydrate content and further acts as a self antigen with the disadvantage of inducing immunological tolerance.

It is therefore the object of the present invention to overcome the above-mentioned problems. In particular it is the object of the present invention to provide a vaccine that may be produced at reasonable manufacturing costs. It is another object of the present invention to induce a long-lasting antibody response with a high immunogenicity of the vaccine and to circumvent or break tolerance mechanisms towards self tissue.

These objects may be solved by the present invention. The present invention relates to a vaccine against cancerous diseases associated with the carcinoembryonic antigen CEA, which comprises at least one CEA mimotope with a length of 6 to 25 amino acids that is recognized immunologically by the monoclonal antibody Col-1.

Thereby, the term mimotope relates to an oligopeptide which mimics at least a part of the extracellular domain of CEA.

Preferably, the length of the mimotope, i.e. oligopeptide, is 6 to 25 amino acids.

The inventive vaccine permits active immunization against cancerous diseases associated with CEA. Thus, a prophylaxis can be obtained against cancerous diseases associated with the carcinoembryonic antigen CEA, such as colorectal carcinoma, some breast cancers, lung, esophagus, thyroid and pancreas carcinoma. In addition, the inventive vaccine can be used to treat an existing cancerous disease or to accompany conventional cancer treatments. Application of the inventive vaccine can completely or partly avoid the considerable disadvantages of conventional cancer treatments such as chemo- or radiotherapy.

Moreover, by active immunization with a mimic according to the present invention, self tolerance against the self-antigen CEA can be broken rather than with identical structures.

As shown below, the inventive vaccine shows a high specific cytotoxicity against tumour cells that is dependent on the vaccine's concentration. Furthermore, it could be demonstrated that the tumour growth in animals could be specifically inhibited.

The inventive vaccine comprises a CEA mimotope which is recognized immunologically by the monoclonal antibody Col-1. According to the invention, mimotopes are antigen surrogates for the induction and amplification of effective immune responses towards CEA. One possibility to select respective CEA mimotopes, i.e. oligopeptides, is to use the monoclonal antibody Col-1 directed against the extracellular domain of CEA. The technology is based on the selection of phage-displayed mimotopes from phage libraries using antibodies against CEA, such as Col-1.

Phage libraries contain a huge repertoire of peptide ligands. The libraries exemplarily used in the present invention were displaying nonameric peptides in linear form or decameric circular peptides, where peptide inserts are flanked by two cysteins allowing a disulfide bond and circularisation. Both libraries were provided by Prof. L. Mazzucchelli (L. Mazzucchelli et al., Blood 1999; 93; 1738-48).

Preferably, the vaccine is phage-free. That is, even if phage-presented oligopeptides with the desired length of 6 to 25 amino acids are used for selecting an effective amino acid sequence with the aid of antibodies acting against CEA, these phage-presented peptides should not be processed into a vaccine but be previously freed from the phage fraction and only then processed to a vaccine employable in particular for humans.

Moreover, the mimotope may be synthesized chemically or genetically.

Preferably, the CEA mimotope is a linear or a cyclic oligopeptide, having the length of 6 to 25 amino acids. Preferably, cyclisation is obtained via disulfide bond formation between two cysteins.

Further, the vaccine of the present invention preferably comprises an active ingredient which displays or presents at least one CEA mimotope once or multiple times. It is preferred that the active ingredient displays or presents at least one CEA mimotope multiple times, for instance, two, three, four, five, six, seven, eight, nine, ten or more times.

Moreover, it is preferred that the CEA mimotope is coupled to a carrier. The mimotope oligopeptides or combinations thereof can be fused or chemically coupled to a carrier to enhance their antigenic density and, therefore, immunogenicity.

It is also preferred that the carrier presents the mimotope in a high density, this means that the mimotope is responsible for the immune reaction. Hence, it is preferred that the carrier presents the CEA mimotope for e.g. twenty, fifty or more times.

Thereby, the carrier should be phage-free and be harmless to humans. The carrier may be immunogenic, however, this is not a necessity.

In the present invention every carrier known in the art may be used. However, preferably, the carrier is selected from the group consisting of keyhole limpet hemocyanin (KLH), tetanus toxoid (TT), cholera toxin subunit B (CTB), polyglycol, like polyethylengycol, poly-lactic acid (PLA), poly-lactic-co-glycolic acid (PLGA), liposome, chitosome, bacterial ghosts, lysine dendrimers, virosomes or their like.

Lysine dendrimers according to the invention are molecules with a tree-like structure whereby the branching is formed of repetitive lysine units. However, the lysine dendrimer may not exclusively consist of lysine units only, but may also involve other units as linkers such as 1,6-hexandiamine or dithioacetylhexandiamine between two lysine branches.

For instance, a lysine dendrimer may have the following structure:

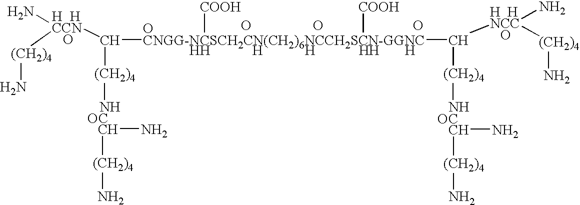

or more schematically:

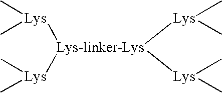

whereby every terminal lysine provides two amino groups that may be used for the coupling of a mimotope either with or without a linker. Naturally, other linkers between the lysine branches as the one shown above are possible.

Moreover, the dendrimer may have a structure as follows:

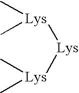

whereby again every terminal lysine provides two amino groups that may be used for coupling of a mimotope either with or without a linker.

In one embodiment of the invention, the mimotope is coupled to the carrier via a linker. Thereby, the linker acts as a spacer that confers flexibility or, if desired, rigidity of the displayed mimotope. The chemical nature of the spacer may vary, depending on the reactivity of the functional groups of the carrier and the mimotope, respectively, and depending on the necessity in respect of flexibility or rigidity. As an example, spacing sequences such as (GP)ₓ, or (G)ₓ, may be mentioned. However, also other reagents such as first acetylation of the lysine amino groups with iodoacetic acid, followed by reaction of the iodine with a mercapto group for instance of cysteine or 3-mercapto-propionic acid, may be mentioned. Also, combinations thereof are possible.

Multiple antigenic peptides (MAPs), containing a lysine dendrimer as a carrier, can be synthesized straight forward if the mimotope peptides are linear. In case of circular oligopeptides, mimotopes can be synthesized and constrained first and, in a second step, coupled chemically to lysine. It is technically important that mimotopes have to adapt the same orientation when displayed on the carrier as by the phage during selection with an antibody, which is a C-terminal coupling to the N-terminus of the phage protein.

As an example, the following schematic build-up of multiple antigen peptides (MAPs) bearing linear and cyclic mimotopes may be used:

First, a multiple antigenic mimotope (MAM) is synthesised, bearing four linear mimotopes bound with or without a spacer or linker such as GG to a lysine dendrimer. Then, 1,6-hexandiamine is acetylated with iodo-acetic acid and subsequently reacted with the MAM as described above:

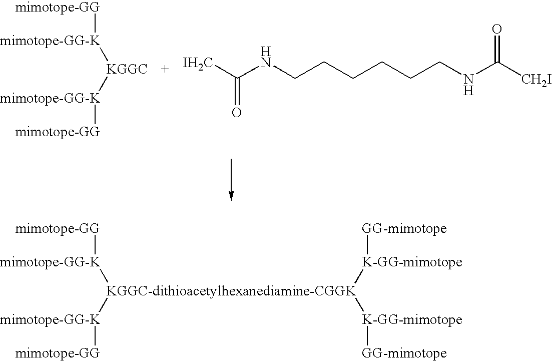

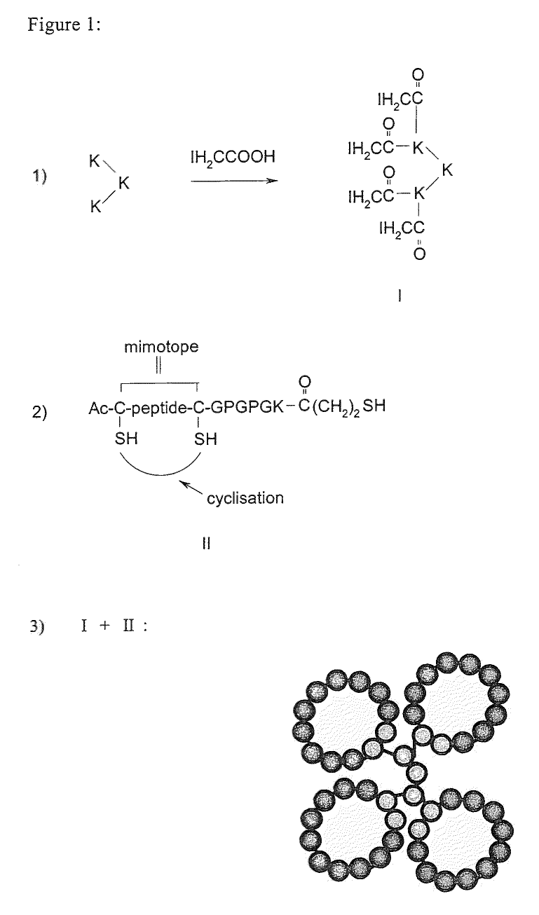

Regarding the multiple antigenic peptides bearing cyclic mimotopes, the following reaction scheme as shown in FIG. 1 may be used.

Thereby, a branched trimeric lysine is reacted with iodo-acetic acid to give product I. Further, the mimotope oligopeptide sequence is synthesized at first as a linear sequence, containing the spacer or linker sequence GPGPGK. Then, the two mercapto groups of the cystein residues are reacted via oxidative formation of a disulfide to give the cyclic mimotope. Afterwards, the lysine residue at the C-terminal of the peptide is reacted with 3-mercapto-propionic acid to give product II, which is subsequently reacted with the activated compound I to give the multiple antigenic peptide containing four cyclic mimotope components as shown schematically in FIG. 1.

It is also possible that the multiple antigenic mimotope (MAM) has the following structure:

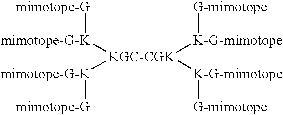

Circular peptides may be preferentially recognized by antibodies preferring conformational epitopes. In contrast, linear peptides are more easily produced synthetically.

Preferably, the CEA mimotope is an oligopeptide with an amino acid sequence selected from the sequences:

DRGGLFRKG
DKGGLLRM
DKGGLMKTI
DKGGLMKTN
DLGGFFKSA
DLGGLVKGN
DLGGLWKMT
DMGGLFRKG
DMGGLWKMV
DQGGLVKQK
DRGGLWKTP
ERAQIIWRG
WDRGLLIKF
C-DRGGLWRTPR-C
C-DSNRGGLWRK-C
C-SNRGGLWRK-C
C-EGRDLGGLLR-C
C-EKWMRASGVA-C
C-ERDRGGLMRR-C
C-FGASGLWKRR-C
C-GNRDQGGLFR-G
C-GPRDRGGLIK-C
C-KDLGGLVKRR-C
C-LWRGGPPAIE-C
C-QRDLGGLRR-C
C-QSMNRGGLWR-C
C-RKWDPGLLGR-C
C-RLALGDAKKY-C
C-SKGGLHKWRH-C
C-SLAIGEFSKK-C
C-TRDLGGLFRD-C
C-VRKGGLIKGR-C

and/or a functional peptide variant of these amino acid sequences that can be obtained by conservative substitution, addition and/or omission of one or more amino acids, preferably one to 50% of mimotope containing amino acids, of these amino acid sequences without changing, i.e. negatively affecting, the binding properties of the sequence to the antibody.

Preferably, the CEA mimotope is an oligopeptide with the following sequences:

DKGGLMKTN
DMGGLFRKG
DRGGLWKTP
C-DSNRGGLWRK-C
C-GPRDRGGLIK-C
C-RLALGDAKKY-C
C-VRKGGLIKGR-C

and/or a functional peptide variant of these amino acid sequences that can be obtained by conservative substitution, addition and/or omission of one or more amino acids, preferably one to 50% of mimotope containing amino acids, of these amino acid sequences without changing, i.e. negatively affecting, the binding properties of the sequence to the antibody.

It is a further embodiment of the invention to provide a process for producing a CEA mimotope by biopanning of phage libraries displaying oligopeptides. Thereby, the length of the oligopeptides is from 6 to 25 amino acids. The conformation of the oligopeptides may be linear or circular.

Further, it is an embodiment of the invention to provide a process for producing a vaccine which comprises as an active ingredient a carrier on which one or more CEA mimotopes are coupled.

The inventive vaccine may further contain promiscuous T-cell epitope peptides, interleukins like e.g. IL-2, IL-4, IL-12, IL-13; INF-gamma, aluminium hydroxid and all other adjuvant known in the art.

In general, by applying the vaccine via different routes, i.e. intramuscular, intradermal, subcutaneous, mucosal or oral, distinct antibody classes, i.e. IgG, IgE, IgA and/or IgM, can be induced towards CEA through the vaccine. Each antibody class takes advantage of a different spectrum of effector mechanisms, IgG and IgA may induce ADCC reactions, IgG subclasses 1 to 3 may induce CDC, IgE antibodies interact with cells bearing the high affinity IgE receptor FcεRI (mast cells, basophils, eosinophils).

The application of the vaccine may be with or without additional adjuvants like Al(OH)₃ or acid-neutralizing or acid-suppressing medications (sucralfate, antacids, H2-receptor blockers, proton pump inhibitors) when oral application is planned.

The CEA mimotope may of course also be used as a diagnostic means for instance in order to test the success of a vaccination. When it is used for diagnostic tests, it is preferably either coupled to carriers which are not immunogenic or which do not interfere with the immunogenicity of the correspondent vaccine used.

Without any restriction to the following examples and figures, the present invention may be exemplified as follows:

FIG. 1: Multiple antigenic peptide containing four cyclic mimotope components

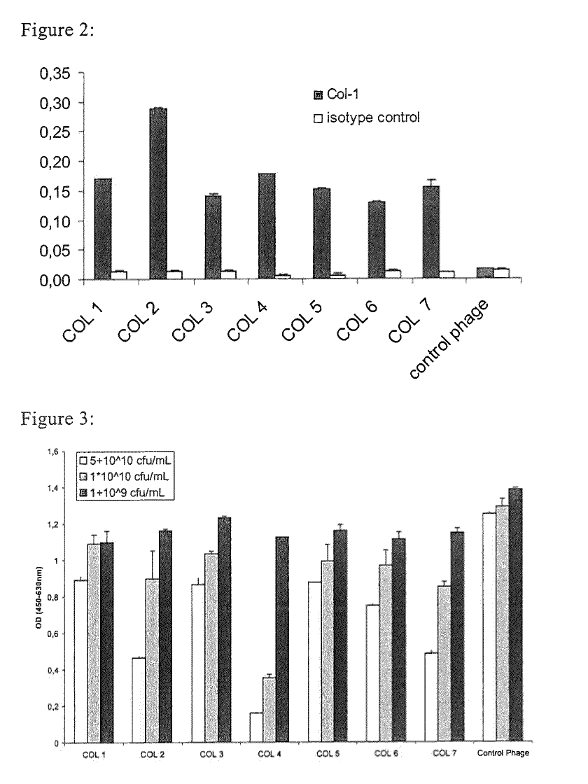

FIG. 2: Specificity ELISA of phage clones. In a sandwich assay, phage clones were bound by coated anti-CEA antibody Col-1 (black columns) and detected by rabbit anti-phage antibody, peroxidase-labelled. No phage binding occurred to isotype control antibody (white columns). X-axis: clone names; Y-axis: signal intensity at OD_{450-630 nm}.

FIG. 3: Mimicry analysis in ELISA competition assay. Coated CEA antigen is detected by Col-1 antibody, rendering a maximal signal of 1,4. Simultaneous incubation was done with titrated phage clones (white columns highest, grey: medium, black: least concentration of phage clones). Bound Col-1 was detected with anti-mouse IgG-peroxidase labelled. X-axis: clone names, Y-axis: colour intensity at OD_{450-630 nm}.

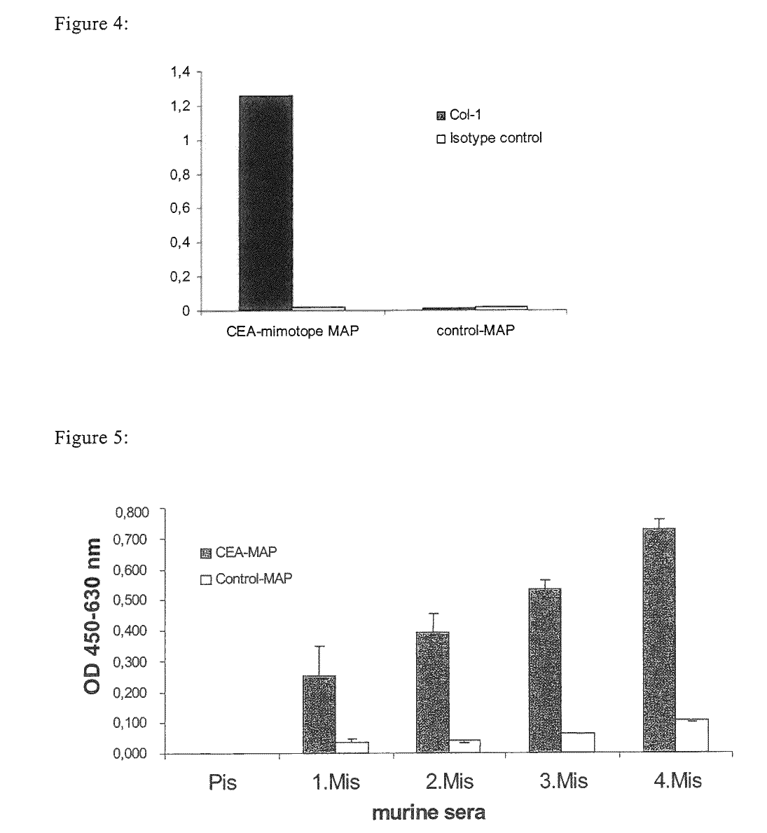

FIG. 4: Antigenicity check of an octameric mimotope-MAP in ELISA. MAPs were coated and incubated with Col-1 (black columns) or isotype control (white columns). Bound antibody was detected by peroxidase-labelled anti-mouse antibody. X-axis: substances coated onto ELISA plate. Y-axis: OD_{450-630 nm}

FIG. 5: Specific immunogenicity of CEA-mimotope MAP in BALB/c mice. Sera of immunized mice were tested for binding to the immunogen CEA-mimotope MAP (black columns and to the irrelevant control MAP (white columns). Sera were diluted 1:100, tested individually and bound IgG detected by peroxidase-labelled anti-mouse IgG antibody. The mean values of eight sera±STDEV is shown. PIS: mouse preimmune serum, MIS: mouse immune serum taken during the immunization period. Background reactivities were subtracted. Y-axis: signal intensity.

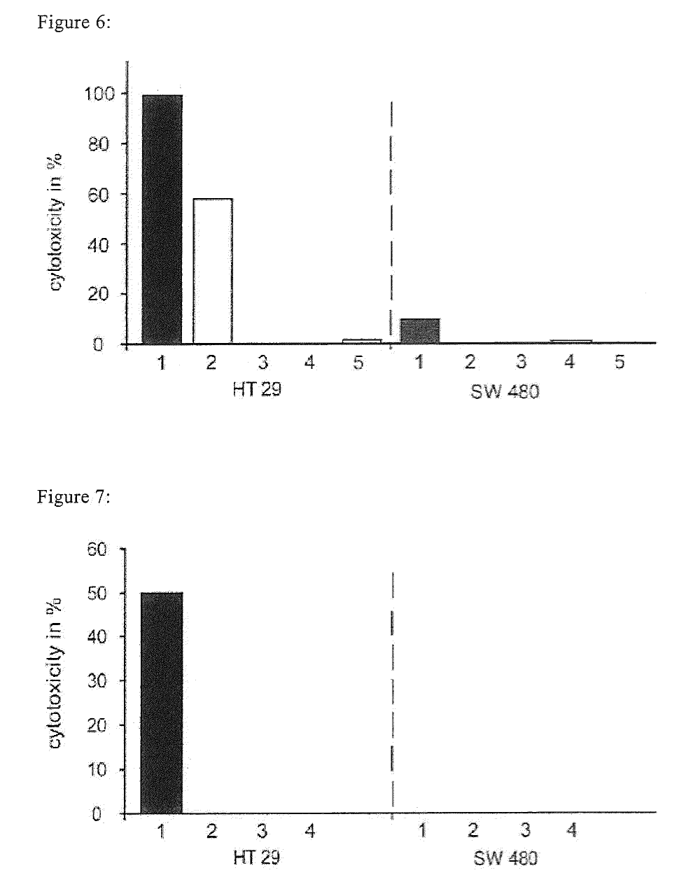

FIG. 6: CDC reaction in vitro. Effects of the mimotope induced antibodies in mediating complement-dependent cytotoxicity. The reaction was determined against the CEA positive cell line HT 29 and against the CEA negative cell line SW 480. Mouse immune sera in different concentrations were tested on the two cell lines. Sera from CEA-MAM immunized mice were used 1:50 (black columns; 1) and 1:100 (white column; 2). The antibody Col-1 (3), the isotype control antibodies IgG2a (4) and IgM (5) were used as negative controls.

FIG. 7: ADCC reaction in vitro. Effects of the antibody-dependent cytotoxicity. The reaction was determined against the CEA positive cell line HT 29 and against the CEA negative cell line SW 480. The CEA-MAP serum was used 1:50 (black columns; 1). The mice immunized with a control-MAP (2) or alum alone (3) and the Col-1 antibody (4) were used as negative controls.

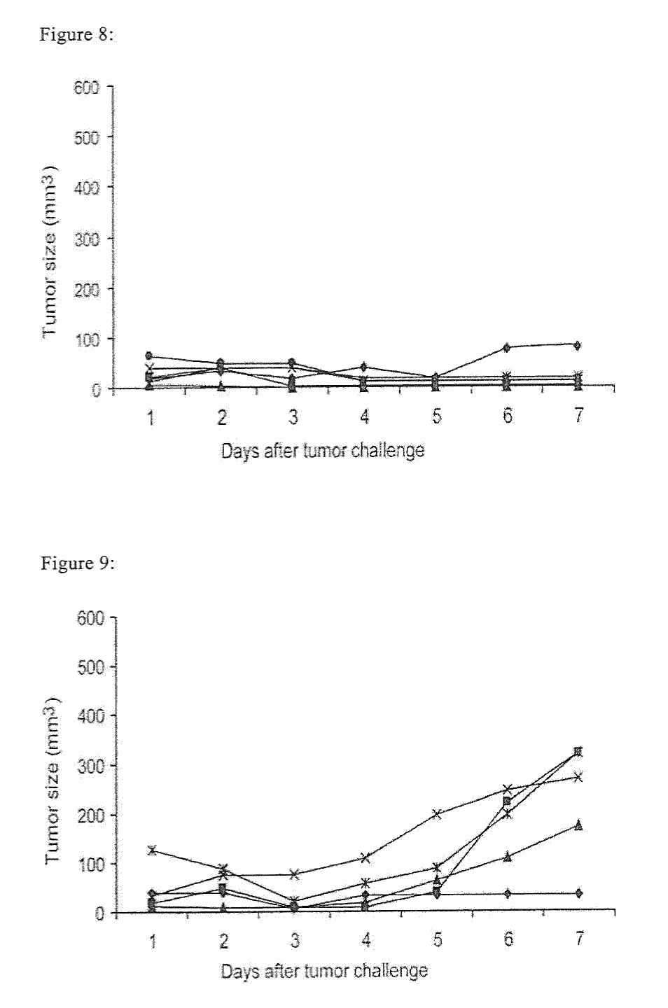

FIG. 8: Anti-tumour activity in CEA mimotope immunized mice. BALB/c mice were immunized with the CEA-MAM. After transplanting Meth-A/CEA tumour cells the tumour size was controlled on daily basis until a tumour volume of 300 mm³ in the non-immunized group was reached. The diagram shows the volume of tumour development (y-axis) during the time course of one week (x-axis).

FIG. 9: Development of tumour growth in BALB/c mice that were immunized with an irrelevant control mimotope. After transplanting Meth-A/CEA tumour cells the tumour size was controlled on daily basis until a tumour volume of 300 mm³ in the non-immunized group was reached. The diagram shows the volume of tumour development (y-axis) during the time course of one week (x-axis).

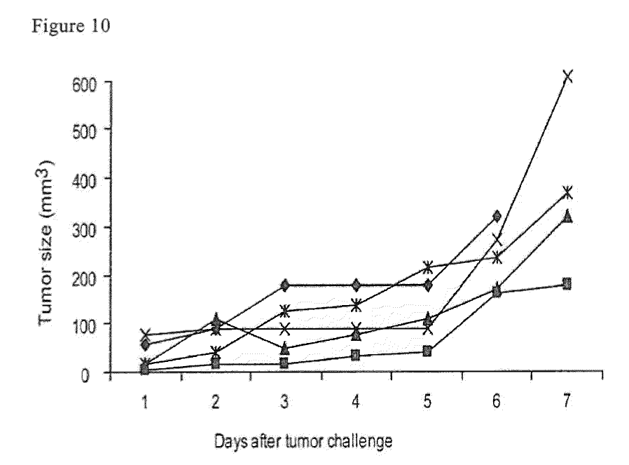

FIG. 10: Development of tumour growth in non immunized BALB/c mice. After transplanting Meth-A/CEA tumour cells the tumour size was controlled on daily basis until a tumour volume of 300 mm³ in the non-immunized group was reached. The diagram shows the volume of tumour development (y-axis) during the time course of one week (x-axis).

### BIOPANNING

Peptide mimotopes were generated using monoclonal antibody Col-1 (Zymed Lab., San Francisco, Calif.) recognizing CEA and being applied in histopathology. For biopannings, an ELISA plate was coated according to standard methods using Col-1. Phages of the amplified libraries displaying linear or constrained peptides were pooled to equal parts and incubated to the coated Col-1. Whereas mimotopes ligands bound to Col-1 unbound phages could be washed away. Bound phages were eluted by low pH incubation, followed by immediate neutralization. In a next step eluted phages are amplified in E. coli and applied for the next round. Four rounds in all were performed.

### Selection of Phages by Colony Screening

After the panning rounds, the amplification of specific ligands was approved by an increase of phage titers. Phages from rounds 3 and 4 were cloned and subjected to colony screening assay using mouse monoclonal IgG₂ₐ antibody Col-1 and an isotype control antibody (mouse IgG_{2a, kappa}; murine myeloma, Sigma) for detection.

### Mimotope Sequences

DNA-sequencing rendered the following aa-sequences from library LL9 displaying linear nonameric peptides (due to failure in the library, also octamers are derived):

DRGGLFRKG
DKGGLLRM
DKGGLMKTI
DKGGLMKTN (clone COL1)
DLGGFFKSA
DLGGLVKGN
DLGGLWKMT
DMGGLFRKG (clone COL3)
DMGGLWKMV
DQGGLVKQK
DRGGLWKTP (clone COL2)
ERAQIIWRG
WDRGLLIKF

From the decameric constrained peptides the following sequences were selected—each flanked by cysteins:

C-DRGGLWRTPR-C
C-DSNRGGLWRK-C (clone COL7)
C-SNRGGLWRK-C
C-EGRDLGGLLR-C
C-EKWMRASGVA-C
C-ERDRGGLMRR-C
C-FGASGLWKRR-C
C-GNRDQGGLFR-C
C-GPRDRGGLIK-C (clone COL6)
C-KDLGGLVKRR-C
C-LWRGGPPAIE-C
C-QRDLGGLRR-C
C-QSMNRGGLWR-C
C-RKWDPGLLGR-C
C-RLALGDAKKY-C (clone COL4)
C-SKGGLHKWRH-C
C-SLAIGEFSKK-C
C-TRDLGGLFRD-C
C-VRKGGLIKGR-C (clone COL5)

### Specificity ELISA

Several mimotopes were selected due to good performance in previous test for further studies, these were the mimotopes, termed COL1-COL7: COL1: DKGGLMKTN; COL2: DRGGLWKTP; COL3: DMGGLFRKG; COL4: C-RLALGDAKKY-C; COL5: C-VRKGGLIKGR-C; COL6: C-GPRDRGGLIK-C; COL7: C-DSNRGGLWRK-C. These clones were amplified, diluted to equal phage particle concentration and further tested for specificity and binding strength to CEA in an ELISA assay (FIG. 2). Here, Col-1 or the isotype control antibody were coated and incubated with amplified phage clones. Bound phage was detected by rabbit anti-phage antibody, which was peroxidase-labeled. After substrate addition and development, the signal intensity was determined in an ELISA reader at OD_{450-630 nm}. Clones COL1-COL7, but not wild type phage without displaying a peptide, were bound specifically by antibody Col-1. No reactivity was observed with the isotype control.

### Mimicry Test

To prove the mimicry potential of selected phage-mimotopes with the original antigen CEA a competitive ELISA assay was performed (FIG. 3). The CEA antigen (human purified; Sigma, St. Louis) was used for coating ELISA plates. After blocking and washing, mimotopes phages were added to wells in three concentrations (5*10¹⁰, 1*10¹⁰, 1*10⁹ particles per ml) simultaneously with antibody Col-1. In a final step, bound Col-1 antibody was detected by a peroxidase-labeled anti-mouse antibody. TMB substrate (BD Biosciences, San Diego, Calif.) was added for development of the colour and signal intensity measured in ELISA reader at OD₄₅₀₋₆₃₀. The reduction of the signal can be interpreted as a competition of the phage-displayed mimotopes with CEA for binding to anti-CEA antibody Col-1. The assay shows 1) that the competition is dose dependent: Higher amounts of phages (white columns) have higher capabilities for competition; 2.) the competition is specific: A control phage displaying an irrelevant peptide does not compete with CEA, even at the highest dose. 3.) Moreover, depending on their sequence, the mimotopes displayed distinct competition potential with CEA, with clone COL4 being the best candidate. This assay evidenced that selected mimotopes are mimics of the Col-1 epitope on CEA antigen.

### Synthetic Production of Mimotopes in Map Configuration

A sequence DRGGLWKTP of linear mimotope clone COL2 was selected for synthetic production of the multiple antigenic peptide

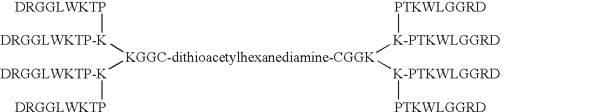

(piChem, Graz, Austria). The correct fold of the MAP was controlled via Col-1 binding analysis in ELISA. FIG. 4 shows that the coated MAP is specifically recognized by Col-1, but not by isotype control antibody.

For control an irrelevant linear MAP

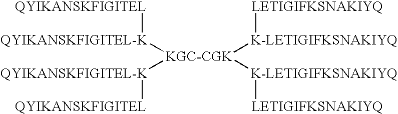

was chosen and was not recognized by either antibody.

### Immunization Experiments in BALB/c Mice

### Synthetic CEA-mimotope MAP

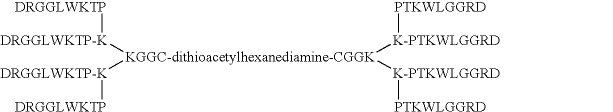

was diluted to 1 mg/ml PBS, and 100 μg in 50 μl per dose were applied intraperitoneally to BALB/c mice (n=8) using 100 μl Al(OH)₃ as adjuvans. Immunizations were performed four times in 14-days intervals. Serum was taken from the tail vein before treatments (pre-immune serum; PIS), and 10 days after each immunization (mouse immune serum, MIS) and the IgG titers monitored (FIG. 5). Sera were tested for reactivity towards the CEA-mimotope MAP, or an irrelevant MAP

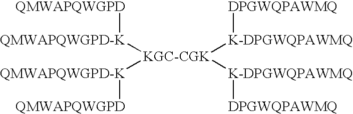

Both antigens, MAM and control-MAP, were coated to ELISA plates, blocked and incubated in duplicates with individual mouse sera, diluted 1:100 in blotting buffer. After washing, bound antibodies were detected by peroxidase-labeled anti-mouse IgG antibody. FIG. 5 shows that an increase of IgG titers towards the CEA-mimotope MAP, but not towards the control MAP was observed in all 8 mice during the immunization period. From our experiments it can be concluded that the mimotopes do mimic epitopes of CEA and are specifically immunogenic.

### Complement and Antibody-Dependent Cytotoxicity Assay

Complement-dependent cytotoxicity (CDC) and antibody-dependent-cytotoxicity (ADCC) effectivity of the antibodies induced by mimotope vaccination were measured with the CytoTox 96 Nonradioactive Cytotoxicity assay (Promega, Madison, Wis.). HT29 CEA overexpressing cells were used as positive target cells. SW480 CEA-negative colon cancer cells served as a negative target control cell line. The number of both target cells was optimized to 2×10⁵ cells/ml. For CDC reactions (FIG. 6), pooled fifth immune sera were diluted 1:50 (1) or 1:100 (2) in CytoTox 96 assay medium. Additionally, the antibody Col-1 (3), an IgG2a (4) and an IgM (5) antibody (Sigma, Vienna, Austria) served as negative controls. Spleen cells of naïve BALB/c mice were prepared by mashing the spleen and lysing the erythrocytes with ammonium chloride and used as effector cells. For the ADCC assay, pooled fifth mouse immune sera diluted 1:50 (1) of the mimotope-immunized mice were used in FIG. 7. As controls, the pooled fifth control-MAP serum (2), the sear from mice immunized with alum alone (3) and the antibody Col-1 (4) were used. All assay procedures and readouts were done as described in the manufacturers description. Assays were performed in triplicates. The results of the cytotoxicity was calculated as follows:

$$\% cytotoxicity = \frac{\begin{matrix}experimental - \\ effector spontaneous - target spontaneous\end{matrix}}{target maximum - target spontaneous} \times 100$$

The highest value was corrected to 100% and the other samples were adjusted respectively. The immune sera of mimotope-vaccinated mice in the CDC assay (FIG. 6) and the CEA-MAP serum in the ADCC assay (FIG. 7) shows a specific concentration dependent increase of cytotoxicity on CEA overexpressing cells in comparison to the negative controls. The CDC reaction with the serum dilution 1:50 could achieve 100% cytotoxicity, the serum diluted 1:100 achieved 51%. The antibodies of the CEA mimotope immunized mice showed 50% cytotoxicity against the CEA overexpressing cell line in the ADCC reaction. Specifity could be demonstrated because neither the irrelevant mimotope immunized group nor the naïve control group were able to elicit an ADCC reaction. In addition, no reaction could be seen on CEA negative SW480 cells.

### Tumour Cell Injection and Histopathology

Meth-A/CEA tumour cells were cultured in RPMI 1640 medium with 10% heat inactivated fetal calf serum (PAA Laboratories, Austria), 2 mM L-glutamine, 100 U/ml penicillin, 100 μg/ml streptomycin, non-essential amino acids and 1 mM sodium pyruvate (GIBCO/Invitrogen, Austria). Cells were loosened with Na-EDTA. 10⁷ tumour cells/ml were washed three times in phosphate-buffered saline (PBS) and 50 μl of the cell suspension with the indicated cell number was injected subcutaneously into the shaved right flank of the mice. Experimental groups consisted of 4-6 mice. Tumour development was followed by serial measurements of tumour size, the tumour volume was calculated according to the equation: tumour volume (mm³)=d²×D/2, where d was the shortest and D the longest diameter. FIG. 8 shows that over a period of 7 days the tumour growth stagnated within BALB/c mice immunized with CEA-MAM in contrast to tumours within BALB/c mice immunized with irrelevant control mimotope (FIG. 9) and in non immunized mice (FIG. 10).

Animals were euthanized when the tumour reached a volume of 300 mm³. Tumour sections were fixed in 10% buffered formalin, processed, and embedded in paraffin. 4 μm sections were HE stained and examined in a light microscope (Olympus BH2). Micrographs were taken at a magnification of 100× and 400× using an Olympus digital camera indicating that the mimotope vaccine inhibits the settling of Meth-A/CEA cells through inflammation, whereas sham or non-treated animals show flourishing tumour cell proliferation (data not shown).

### Clinical Impact of the Invention

An impressive number of the tumours with the highest prevalence, including colon cancer, show CEA-overexpression. Vaccination against this target will induce antibodies exerting diverse anti-tumour effects, depending on their isotype. Therefore, mimotopes as antigen surrogates of CEA would alone, or in an adjuvant setting, activate the immune system of tumour patients to react towards CEA as self antigen.

1. A vaccine against cancerous diseases associated with the carcinoembryonic antigen CEA, comprising at least one CEA mimotope with a length of 6 to 25 amino acids that is recognized immunologically by the monoclonal antibody Col-1.

2. A vaccine according to claim 1, characterized in that the CEA mimotope is a linear or a cyclic oligopeptide.

3. A vaccine according to claim 1, comprising an active ingredient which displays at least one CEA mimotope once or multiple times.

4. A vaccine according to claim 1, wherein the CEA mimotope is coupled to a carrier.

5. A vaccine according to claim 4 1, wherein the CEA mimotope is coupled to a carrier and the carrier is selected from the group consisting of keyhole limpet hemocyanin (KLH), tetanus toxoid (TT), cholera toxin subunit B (CTB), polyglycol, like polyethylengycol, poly-lactic acid (PLA), poly-lactic-co-glycolic acid (PLGA), liposome, chitosome, bacterial ghosts, lysine dendrimers and virosomes.

6. A vaccine according to claim 4, wherein the CEA mimotope is conjugated to the carrier via a linker.

7. A vaccine according to claim 1, wherein the at least one CEA mimotope has an amino acid sequence selected from the sequences consisting of:

| | |
|---|---|
| DRGGLFRKG | (SEQ ID NO: 1) |
| DKGGLLRM | (SEQ ID NO: 2) |
| DKGGLMKTI | (SEQ ID NO: 3) |
| DKGGLMKTN | (SEQ ID NO: 4) |
| DLGGFFKSA | (SEQ ID NO: 5) |
| DLGGLVKGN | (SEQ ID NO: 6) |
| DLGGLWKMT | (SEQ ID NO: 7) |
| DMGGLFRKG | (SEQ ID NO: 8) |
| DMGGLWKMV | (SEQ ID NO: 9) |
| DQGGLVKQK | (SEQ ID NO: 10) |
| DRGGLWKTP | (SEQ ID NO: 11) |
| ERAQIIWRG | (SEQ ID NO: 12) |
| WDRGLLIKF | (SEQ ID NO: 13) |
| C-DRGGLWRTPR-C | (SEQ ID NO: 14) |
| C-DSNRGGLWRK-C | (SEQ ID NO: 15) |
| C-SNRGGLWRK-C | (SEQ ID NO: 16) |
| C-EGRDLGGLLR-C | (SEQ ID NO: 17) |
| C-EKWMRASGVA-C | (SEQ ID NO: 18) |
| C-ERDRGGLMRR-C | (SEQ ID NO: 19) |
| C-FGASGLWKRR-C | (SEQ ID NO: 20) |
| C-GNRDQGGLFR-C | (SEQ ID NO: 21) |
| C-GPRDRGGLIK-C | (SEQ ID NO: 22) |
| C-KDLGGLVKRR-C | (SEQ ID NO:23) |
| C-LWRGGPPAIE-C | (SEQ ID NO: 24) |
| C-QRDLGGLRR-C | (SEQ ID NO: 25) |
| C-QSMNRGGLWR-C | (SEQ ID NO: 26) |
| C-RKWDPGLLGR-C | (SEQ ID NO: 27) |
| C-RLALGDAKKY-C | (SEQ ID NO: 28) |
| C-SKGGLHKWRH-C | (SEQ ID NO: 29) |
| C-SLAIGEFSKK-C | (SEQ ID NO: 30) |
| C-TRDLGGLFRD-C; | (SEQ ID NO: 31) |
| and | |
| C-VRKGGLIKGR-C. | (SEQ ID NO: 32) |

or a functional peptide variant of these amino acid sequences that can be obtained by conservative substitution, addition and/or omission of one or more amino acids of these amino acid sequences without negatively affecting the binding properties of the sequence to the antibody.

8. A CEA mimotope, characterized in that it is recognized immunologically by the monoclonal antibody Col-1 and comprises an oligopeptide with a length of 6 to 25 amino acids.

9. A CEA mimotope according to claim 8, wherein the one oligopeptide has an amino acid sequence selected from the sequences the sequences consisting of:

| | |
|---|---|
| DRGGLFRKG | (SEQ ID NO: 1) |
| DKGGLLRM | (SEQ ID NO: 2) |
| DKGGLMKTI | (SEQ ID NO: 3) |
| DKGGLMKTN | (SEQ ID NO: 4) |
| DLGGFFKSA | (SEQ ID NO: 5) |
| DLGGLVKGN | (SEQ ID NO: 6) |
| DLGGLWKMT | (SEQ ID NO: 7) |
| DMGGLFRKG | (SEQ ID NO: 8) |
| DMGGLWKMV | (SEQ ID NO: 9) |
| DQGGLVKQK | (SEQ ID NO: 10) |
| DRGGLWKTP | (SEQ ID NO: 11) |
| ERAQIIWRG | (SEQ ID NO: 12) |
| WDRGLLIKF | (SEQ ID NO: 13) |
| C-DRGGLWRTPR-C | (SEQ ID NO: 14) |
| C-DSNRGGLWRK-C | (SEQ ID NO: 15) |
| C-SNRGGLWRK-C | (SEQ ID NO: 16) |
| C-EGRDLGGLLR-C | (SEQ ID NO: 17) |
| C-EKWMRASGVA-C | (SEQ ID NO: 18) |
| C-ERDRGGLMRR-C | (SEQ ID NO: 19) |
| C-FGASGLWKRR-C | (SEQ ID NO: 20) |
| C-GNRDQGGLFR-C | (SEQ ID NO: 21) |
| C-GPRDRGGLIK-C | (SEQ ID NO: 22) |
| C-KDLGGLVKRR-C | (SEQ ID NO: 23) |
| C-LWRGGPPAIE-C | (SEQ ID NO:24) |
| C-QRDLGGLRR-C | (SEQ ID NO: 25) |
| C-QSMNRGGLWR-C | (SEQ ID NO: 26) |
| C-RKWDPGLLGR-C | (SEQ ID NO: 27) |
| C-RLALGDAKKY-C | (SEQ ID NO: 28) |
| C-SKGGLHKWRH-C | (SEQ ID NO: 29) |
| C-SLAIGEFSKK-C | (SEQ ID NO: 30) |
| C-TRDLGGLFRD-C; | (SEQ ID NO:31) |
| and | |
| C-VRKGGLIKGR-C | (SEQ ID NO: 32) |

or a functional peptide variant of these amino acid sequences that can be obtained by conservative substitution, addition and/or omission of one or more amino acids of these amino acid sequences without changing the binding properties of the sequence to the antibody.

10. A process for producing a CEA mimotope according to claim 8, which process comprises biopanning of phage libraries displaying oligopeptides.

11. A process for producing a vaccine according to claim 1, which process comprises conjugating one or more CEA mimotopes to a carrier.

12. (canceled)

13. A diagnostic test kit comprising the mimotope of claim 8.


SEQUENCE LISTING (32 sequences)

SEQ ID NO: 1 - protein, 9 aa, Artificial Sequence
  Phage LL9 Library
DRGGLFRKG

SEQ ID NO: 2 - protein, 8 aa, Artificial Sequence
  Phage LL9 Library
DKGGLLRM

SEQ ID NO: 3 - protein, 9 aa, Artificial Sequence
  Phage LL9 Library
DKGGLMKTI

SEQ ID NO: 4 - protein, 9 aa, Artificial Sequence
  Phage LL9 Library
DKGGLMKTN

SEQ ID NO: 5 - protein, 9 aa, Artificial Sequence
  Phage LL9 Library
DLGGFFKSA

SEQ ID NO: 6 - protein, 9 aa, Artificial Sequence
  Phage LL9 Library
DLGGLVKGN

SEQ ID NO: 7 - protein, 9 aa, Artificial Sequence
  Phage LL9 Library
DLGGLWKMT

SEQ ID NO: 8 - protein, 9 aa, Artificial Sequence
  Phage LL9 Library
DMGGLFRKG

SEQ ID NO: 9 - protein, 9 aa, Artificial Sequence
  Phage LL9 Library
DMGGLWKMV

SEQ ID NO: 10 - protein, 9 aa, Artificial Sequence
  Phage LL9 Library
DQGGLVKQK

SEQ ID NO: 11 - protein, 9 aa, Artificial Sequence
  Phage LL9 Library
DRGGLWKTP

SEQ ID NO: 12 - protein, 9 aa, Artificial Sequence
  Phage LL9 Library
ERAQIIWRG

SEQ ID NO: 13 - protein, 9 aa, Artificial Sequence
  Phage LL9 Library
WDRGLLIKF

SEQ ID NO: 14 - protein, 12 aa, Artificial Sequence
  Phage LL9 Library
CDRGGLWRTPRC

SEQ ID NO: 15 - protein, 12 aa, Artificial Sequence
  Phage LL9 Library
CDSNRGGLWRKC

SEQ ID NO: 16 - protein, 11 aa, Artificial Sequence
  Phage LL9 Library
CSNRGGLWRKC

SEQ ID NO: 17 - protein, 12 aa, Artificial Sequence
  Phage LL9 Library
CEGRDLGGLLRC

SEQ ID NO: 18 - protein, 12 aa, Artificial Sequence
  Phage LL9 Library
CEKWMRASGVAC

SEQ ID NO: 19 - protein, 12 aa, Artificial Sequence
  Phage LL9 Library
CERDRGGLMRRC

SEQ ID NO: 20 - protein, 12 aa, Artificial Sequence
  Phage LL9 Library
CFGASGLWKRRC

SEQ ID NO: 21 - protein, 12 aa, Artificial Sequence
  Phage LL9 Library
CGNRDQGGLFRC

SEQ ID NO: 22 - protein, 12 aa, Artificial Sequence
  Phage LL9 Library
CGPRNRGGLIKC

SEQ ID NO: 23 - protein, 12 aa, Artificial Sequence
  Phage LL9 Library
CKDLGGLVKRRC

SEQ ID NO: 24 - protein, 12 aa, Artificial Sequence
  Phage LL9 Library
CLWRGGPPAIEC

SEQ ID NO: 25 - protein, 11 aa, Artificial Sequence
  Phage LL9 Library
CQRDLGGLRRC

SEQ ID NO: 26 - protein, 12 aa, Artificial Sequence
  Phage LL9 Library
CQSMNRGGLWRC

SEQ ID NO: 27 - protein, 12 aa, Artificial Sequence
  Phage LL9 Library
CRKWDPGLLGRC

SEQ ID NO: 28 - protein, 12 aa, Artificial Sequence
  Phage LL9 Library
CRLALGDAKKYC

SEQ ID NO: 29 - protein, 12 aa, Artificial Sequence
  Phage LL9 Library
CSKGGLHKWRHC

SEQ ID NO: 30 - protein, 12 aa, Artificial Sequence
  Phage LL9 Library
CSLAIGEFSKKC

SEQ ID NO: 31 - protein, 12 aa, Artificial Sequence
  Phage LL9 Library
CTRDLGGLFRDC

SEQ ID NO: 32 - protein, 12 aa, Artificial Sequence
  Phage LL9 Library
CVRKGGLIKGRC